(19) United States
(12) Patent Application Publication
(10) Pub. No.: US 2010/0087714 A1
(43) Pub. Date: Apr. 8, 2010

(54) REDUCING CROSS-TALK IN A MEASUREMENT SYSTEM

(75) Inventors: James Watson, Dunfermline (GB); Paul Stanley Addison, Edinburgh (GB)

Correspondence Address: Nellcor Puritan Bennett LLC;ATTN: IP Legal, 6135 Gunbarrel Avenue, Boulder, CO, 80301, US

(73) Assignee: Nellcor Puritan Bennett Ireland, Galway (IE)

(21) Appl. No.: 12/245,459

(22) Filed: Oct. 3, 2008

(51) Int. Cl. A61B 5/00

(52) U.S. Cl. 600/301

(57) ABSTRACT

According to embodiments, techniques for determining one or more physiological characteristics in a measurement system which may include cross-talk are disclosed. A sensor or probe may be used to generate two or more a plethysmograph or photoplethysmograph (PPG) signals from a patient. The obtained signals may include an infrared signal and a red signal, and may be subject to an additional measurement noise. The obtained signal may be combined to form a detected signal. The detected signal may be filtered to partially or fully remove noise. The filtered detected signal may be demodulated to separate the red signal and the infrared signal. The recovered red and infrared signals may be processed by additional filters to partially or fully remove cross-talk. The processed red and infrared signals may then be used to determine physiological characteristics of a patient such as a pulse rate, a respiration rate, and a blood oxygen saturation level using the wavelet transform and/or scalogram of at least one of the processed red and infrared signals. The partial or full removal of cross-talk from the red signal and infrared signal may result in a more reliable determination of physiological characteristics than would be possible in a system in which cross-talk was not removed.

### SUMMARY

The present disclosure relates to signal processing and, more particularly, the present disclosure relates to reducing cross-talk in a signal, such as a photoplethysmograph (PPG) signal, so that characteristics of one or more wavelet scalograms of the signal may be used to determine physiological characteristics of a patient.

In an embodiment, a pulse oximetry system is used to measure physiological characteristics of a patient. The pulse oximetry system may include a sensor that detects two or more wavelengths of radiation at a detector. The detector may generate two or more output signals based on the detected radiation. In an embodiment, the output signals may include an infrared signal and a red signal that correspond to infrared light and red light, respectively, that are detected after passing through the tissue of a patient. In an embodiment, the output signals may be processed to remove cross-talk and produce two or more processed signals.

In an embodiment, processing the output signals may include the steps of summing the output signals and a noise signal to obtain a detected signal, filtering the detected signal to obtain a filtered detected signal, and demodulating the filtered detected signal to obtain two or more recovered signals. In an embodiment, filtering the detected signal may include applying an analog band-pass filter to the detected signal. The analog band-pass filter may be designed to reduce the amount of noise present in the detected signal and may generate cross-talk in the filtered detected signal. In an embodiment, one recovered signal may correspond to a demodulated red signal and another recovered signal may correspond to a demodulated infrared signal. In an embodiment, two or more recovered signals may be filtered to obtain a respective number of processed signals. In an embodiment, filtering each recovered signal may include applying a low-pass filter to each recovered signal. In an embodiment, the low-pass filter applied to each recovered signal may include filter coefficients selected to reduce the amount of cross-talk present in the processed signal. For example, the cross-talk may be reduced using one or more signal processing techniques to partially or fully remove cross-talk in the processed signal.

Continuous wavelet transforms allow for the use of a range of wavelets with scales spanning the scales of interest of a signal such that small scale signal components correlate well with the smaller scale wavelets and thus manifest at high energies at smaller scales in the transform. Likewise, large scale signal components correlate well with the larger scale wavelets and thus manifest at high energies at larger scales in the transform. Thus, components at different scales may be separated and extracted in the transform domain. Moreover, the use of a continuous range of wavelets in scale and time position allows for a higher resolution transform than is possible relative to discrete techniques.

In an embodiment, one or more scalograms may be obtained by processing the wavelet transform corresponding to at least one of the processed output signals. Each scalogram may represent the energy density of the PPG signal, where a suitable scaling has been performed to emphasize certain scale values or ranges of interest for the analysis of the PPG signal. In addition, the scalogram may contain information on the real part of the wavelet transform, the imaginary part of the wavelet transform, the phase of the wavelet transform, any other suitable part of the wavelet transform, or any combination thereof.

In an embodiment, one or more physiological characteristics of the patient may then be determined based on an analysis of one or more scalograms. Physiological characteristics may include a pulse rate, a respiration rate, blood oxygen saturation, and any other suitable physiological characteristic, or any combination thereof. In an embodiment, the partial or full removal of cross-talk from the processed signals may results in more reliable determination of physiological characteristics than would be possible in a system in which cross-talk was not removed.

### BRIEF DESCRIPTION OF THE DRAWINGS

The above and other features of the present disclosure, its nature and various advantages will be more apparent upon consideration of the following detailed description, taken in conjunction with the accompanying drawings in which:

FIG. 1 shows an illustrative pulse oximetry system in accordance with an embodiment;

FIG. 2 is a block diagram of the illustrative pulse oximetry system of FIG. 1 coupled to a patient in accordance with an embodiment;

FIGS. 3(a) and 3(b) show illustrative views of a scalogram derived from a PPG signal in accordance with an embodiment;

FIG. 3(c) shows an illustrative scalogram derived from a signal containing two pertinent components in accordance with an embodiment;

FIG. 3(d) shows an illustrative schematic of signals associated with a ridge in FIG. 3(c) and illustrative schematics of a further wavelet decomposition of these newly derived signals in accordance with an embodiment;

FIGS. 3(e) and 3(f) are flow charts of illustrative steps involved in performing an inverse continuous wavelet transform in accordance with embodiments;

FIG. 4 is a block diagram of an illustrative continuous wavelet processing system in accordance with an embodiment;

FIG. 5 shows an illustrative module for reducing noise and cross-talk in one or more detected PPG signals in accordance with an embodiment; and

FIG. 6 is a block diagram of an illustrative system for determining physiological characteristics from a signal that had been pre-processed to remove cross-talk in accordance with an embodiment.

### DETAILED DESCRIPTION

An oximeter is a medical device that may determine the oxygen saturation of the blood. One common type of oximeter is a pulse oximeter, which may indirectly measure the oxygen saturation of a patient's blood (as opposed to measuring oxygen saturation directly by analyzing a blood sample taken from the patient) and changes in blood volume in the skin. Ancillary to the blood oxygen saturation measurement, pulse oximeters may also be used to measure the pulse rate of the patient. Pulse oximeters typically measure and display various blood flow characteristics including, but not limited to, the oxygen saturation of hemoglobin in arterial blood.

An oximeter may include a light sensor that is placed at a site on a patient, typically a fingertip, toe, forehead or earlobe, or in the case of a neonate, across a foot. The oximeter may pass light using a light source through blood perfused tissue and photoelectrically sense the absorption of light in the tissue. For example, the oximeter may measure the intensity of light that is received at the light sensor as a function of time. A signal representing light intensity versus time or a mathematical manipulation of this signal (e.g., a scaled version thereof, a log taken thereof, a scaled version of a log taken thereof, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate the amount of the blood constituent (e.g., oxyhemoglobin) being measured as well as the pulse rate and when each individual pulse occurs.

The light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption. Red and infrared wavelengths may be used because it has been observed that highly oxygenated blood will absorb relatively less red light and more infrared light than blood with a lower oxygen saturation. By comparing the intensities of two wavelengths at different points in the pulse cycle, it is possible to estimate the blood oxygen saturation of hemoglobin in arterial blood.

When the measured blood parameter is the oxygen saturation of hemoglobin, a convenient starting point assumes a saturation calculation based on Lambert-Beer's law. The following notation will be used herein:

I(λ,t)=Iₒ(λ)exp(−(sβₒ(λ)+(1−s)βᵣ(λ))l(t)) (1)

where:
λ=wavelength;
t=time;
I=intensity of light detected;
Iₒ=intensity of light transmitted;
s=oxygen saturation;
βₒ, βᵣ=empirically derived absorption coefficients; and
l(t)=a combination of concentration and path length from emitter to detector as a function of time.

The traditional approach measures light absorption at two wavelengths (e.g., red and infrared (IR)), and then calculates saturation by solving for the “ratio of ratios” as follows.
1. First, the natural logarithm of (1) is taken (“log” will be used to represent the natural logarithm) for IR and Red

log I=log Iₒ−(sβₒ+(1−s)βᵣ)l (2)
2. (2) is then differentiated with respect to time

$$\frac{d log I}{d t} = - ( s {β}_{o} + ( 1 - s ) {β}_{r} ) \frac{d l}{d t} \qquad ( 3 )$$

3. Red (3) is divided by IR (3)

$$\frac{d log I ( {λ}_{R} ) / d t}{d log I ( {λ}_{IR} ) / d t} = \frac{s {β}_{o} ( {λ}_{R} ) + ( 1 - s ) {β}_{r} ( {λ}_{R} )}{s {β}_{o} ( {λ}_{IR} ) + ( 1 - s ) {β}_{r} ( {λ}_{IR} )} \qquad ( 4 )$$

4. Solving for s

$$s = \frac{\frac{d log I ( {λ}_{IR} )}{d t} {β}_{r} ( {λ}_{R} ) - \frac{d log I ( {λ}_{R} )}{d t} {β}_{r} ( {λ}_{IR} )}{\begin{matrix}\frac{d log I ( {λ}_{R} )}{d t} ( {β}_{o} ( {λ}_{IR} ) - {β}_{r} ( {λ}_{IR} ) ) - \\ \frac{d log I ( {λ}_{IR} )}{d t} ( {β}_{o} ( {λ}_{R} ) - {β}_{r} ( {λ}_{R} ) )\end{matrix}}$$

Note in discrete time

$$\frac{d log I ( λ , t )}{d t} ≃ log I ( λ , {t}_{2} ) - log I ( λ , {t}_{1} )$$

### Using log A−log B=log A/B,

$$\frac{d log I ( λ , t )}{d t} ≃ log ( \frac{I ( {t}_{2} , λ )}{I ( {t}_{1} , λ )} )$$

So, (4) can be rewritten as

$$\frac{\frac{d log I ( {λ}_{R} )}{d t}}{\frac{d log I ( {λ}_{IR} )}{d t}} ≃ \frac{log ( \frac{I ( {t}_{1} , {λ}_{R} )}{I ( {t}_{2} , {λ}_{R} )} )}{log ( \frac{I ( {t}_{1} , {λ}_{IR} )}{I ( {t}_{2} , {λ}_{IR} )} )} = R \qquad ( 5 )$$

where R represents the “ratio of ratios.” Solving (4) for s using (5) gives

$$s = \frac{{β}_{r} ( {λ}_{R} ) - R {β}_{r} ( {λ}_{IR} )}{R ( {β}_{o} ( {λ}_{IR} ) - {β}_{r} ( {λ}_{IR} ) ) - {β}_{o} ( {λ}_{R} ) + {β}_{r} ( {λ}_{R} )} .$$

From (5), R can be calculated using two points (e.g., PPG maximum and minimum), or a family of points. One method using a family of points uses a modified version of (5). Using the relationship

$$\frac{d log I}{d t} = \frac{d I / d t}{I} \qquad ( 6 )$$

now (5) becomes

$$\begin{matrix}\frac{\frac{d log I ( {λ}_{R} )}{d t}}{\frac{d log I ( {λ}_{IR} )}{d t}} ≃ \frac{\frac{I ( {t}_{2} , {λ}_{R} ) - I ( {t}_{1} , {λ}_{R} )}{I ( {t}_{1} , {λ}_{R} )}}{\frac{I ( {t}_{2} , {λ}_{IR} ) - I ( {t}_{1} , {λ}_{IR} )}{I ( {t}_{1} , {λ}_{IR} )}} \\ = \frac{[ I ( {t}_{2} , {λ}_{R} ) - I ( {t}_{1} , {λ}_{R} ) ] I ( {t}_{1} , {λ}_{IR} )}{[ I ( {t}_{2} , {λ}_{IR} ) - I ( {t}_{1} , {λ}_{IR} ) ] I ( {t}_{1} , {λ}_{R} )} \\ = R\end{matrix} \qquad ( 7 )$$

which defines a cluster of points whose slope of y versus x will give R where

x(t)=[I(t₂,λ_{IR})−I(t₁,λ_{IR})]I(t₁, λ_{R})

y(t)=[I(t₂,λ_{R})−I(t₁,λ_{R})]I(t₁, λ_{IR})

y(t)=Rx(t) (8)

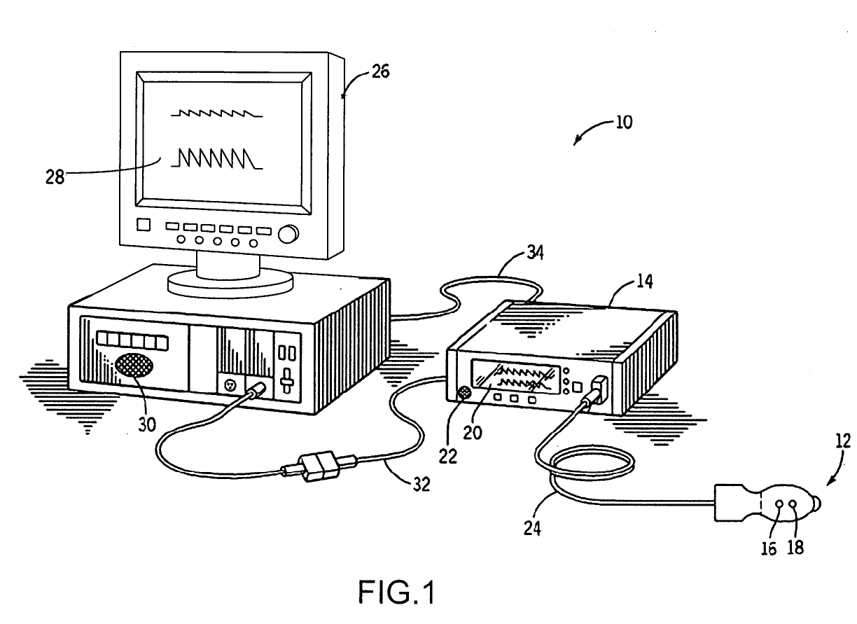

FIG. 1 is a perspective view of an embodiment of a pulse oximetry system 10. System 10 may include a sensor 12 and a pulse oximetry monitor 14. Sensor 12 may include an emitter 16 for emitting light at two or more wavelengths into a patient's tissue. A detector 18 may also be provided in sensor 12 for detecting the light originally from emitter 16 that emanates from the patient's tissue after passing through the tissue.

According to another embodiment and as will be described, system 10 may include a plurality of sensors forming a sensor array in lieu of single sensor 12. Each of the sensors of the sensor array may be a complementary metal oxide semiconductor (CMOS) sensor Alternatively, each sensor of the array may be charged coupled device (CCD) sensor. In another embodiment, the sensor array may be made up of a combination of CMOS and CCD sensors. The CCD sensor may comprise a photoactive region and a transmission region for receiving and transmitting data whereas the CMOS sensor may be made up of an integrated circuit having an array of pixel sensors. Each pixel may have a photodetector and an active amplifier.

According to an embodiment, emitter 16 and detector 18 may be on opposite sides of a digit such as a finger or toe, in which case the light that is emanating from the tissue has passed completely through the digit. In an embodiment, emitter 16 and detector 18 may be arranged so that light from emitter 16 penetrates the tissue and is reflected by the tissue into detector 18, such as a sensor designed to obtain pulse oximetry data from a patient's forehead.

In an embodiment, the sensor or sensor array may be connected to and draw its power from monitor 14 as shown. In another embodiment, the sensor may be wirelessly connected to monitor 14 and include its own battery or similar power supply (not shown). Monitor 14 may be configured to calculate physiological parameters based at least in part on data received from sensor 12 relating to light emission and detection. In an alternative embodiment, the calculations may be performed on the monitoring device itself and the result of the oximetry reading may be passed to monitor 14. Further, monitor 14 may include a display 20 configured to display the physiological parameters or other information about the system. In the embodiment shown, monitor 14 may also include a speaker 22 to provide an audible sound that may be used in various other embodiments, such as for example, sounding an audible alarm in the event that a patient's physiological parameters are not within a predefined normal range.

In an embodiment, sensor 12, or the sensor array, may be communicatively coupled to monitor 14 via a cable 24. However, in other embodiments, a wireless transmission device (not shown) or the like may be used instead of or in addition to cable 24.

In the illustrated embodiment, pulse oximetry system 10 may also include a multi-parameter patient monitor 26. The monitor may be cathode ray tube type, a flat panel display (as shown) such as a liquid crystal display (LCD) or a plasma display, or any other type of monitor now known or later developed. Multi-parameter patient monitor 26 may be configured to calculate physiological parameters and to provide a display 28 for information from monitor 14 and from other medical monitoring devices or systems (not shown). For example, multiparameter patient monitor 26 may be configured to display an estimate of a patient's blood oxygen saturation generated by pulse oximetry monitor 14 (referred to as an “SpO₂” measurement), pulse rate information from monitor 14 and blood pressure from a blood pressure monitor (not shown) on display 28.

Monitor 14 may be communicatively coupled to multi-parameter patient monitor 26 via a cable 32 or 34 that is coupled to a sensor input port or a digital communications port, respectively and/or may communicate wirelessly (not shown). In addition, monitor 14 and/or multi-parameter patient monitor 26 may be coupled to a network to enable the sharing of information with servers or other workstations (not shown). Monitor 14 may be powered by a battery (not shown) or by a conventional power source such as a wall outlet.

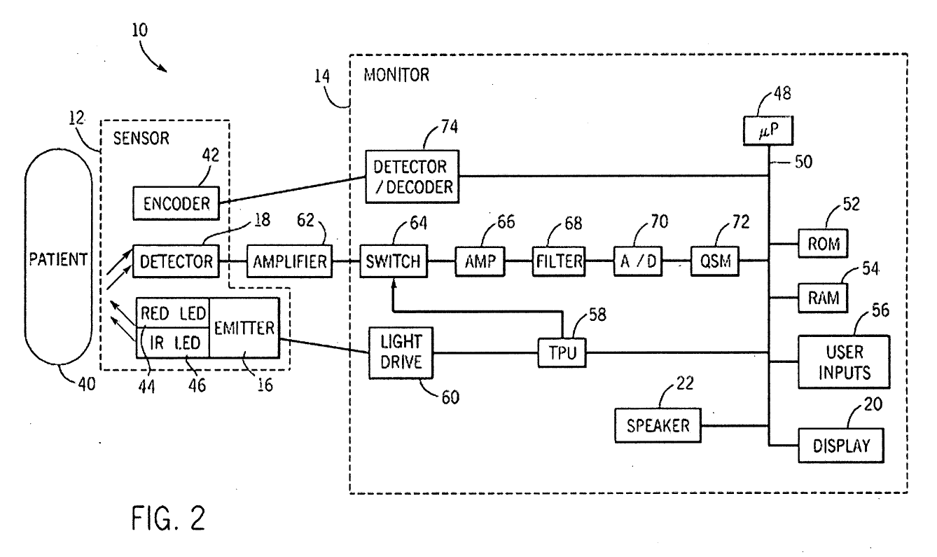

FIG. 2 is a block diagram of a pulse oximetry system, such as pulse oximetry system 10 of FIG. 1, which may be coupled to a patient 40 in accordance with an embodiment. Certain illustrative components of sensor 12 and monitor 14 are illustrated in FIG. 2. Sensor 12 may include emitter 16, detector 18, and encoder 42. In the embodiment shown, emitter 16 may be configured to emit at least two wavelengths of light (e.g., RED and IR) into a patient's tissue 40. Hence, emitter 16 may include a RED light emitting light source such as RED light emitting diode (LED) 44 and an IR light emitting light source such as IR LED 46 for emitting light into the patient's tissue 40 at the wavelengths used to calculate the patient's physiological parameters. In one embodiment, the RED wavelength may be between about 600 nm and about 700 nm, and the IR wavelength may be between about 800 nm and about 1000 nm. In embodiments where a sensor array is used in place of single sensor, each sensor may be configured to emit a single wavelength. For example, a first sensor emits only a RED light while a second only emits an IR light.

It will be understood that, as used herein, the term “light” may refer to energy produced by radiative sources and may include one or more of ultrasound, radio, microwave, millimeter wave, infrared, visible, ultraviolet, gamma ray or X-ray electromagnetic radiation. As used herein, light may also include any wavelength within the radio, microwave, infrared, visible, ultraviolet, or X-ray spectra, and that any suitable wavelength of electromagnetic radiation may be appropriate for use with the present techniques. Detector 18 may be chosen to be specifically sensitive to the chosen targeted energy spectrum of the emitter 16.

In an embodiment, detector 18 may be configured to detect the intensity of light at the RED and IR wavelengths. Alternatively, each sensor in the array may be configured to detect an intensity of a single wavelength. In operation, light may enter detector 18 after passing through the patient's tissue 40. Detector 18 may convert the intensity of the received light into an electrical signal. The light intensity is directly related to the absorbance and/or reflectance of light in the tissue 40. That is, when more light at a certain wavelength is absorbed or reflected, less light of that wavelength is received from the tissue by the detector 18. After converting the received light to an electrical signal, detector 18 may send the signal to monitor 14, where physiological parameters may be calculated based on the absorption of the RED and IR wavelengths in the patient's tissue 40.

In an embodiment, encoder 42 may contain information about sensor 12, such as what type of sensor it is (e.g., whether the sensor is intended for placement on a forehead or digit) and the wavelengths of light emitted by emitter 16. This information may be used by monitor 14 to select appropriate algorithms, lookup tables and/or calibration coefficients stored in monitor 14 for calculating the patient's physiological parameters.

Encoder 42 may contain information specific to patient 40, such as, for example, the patient's age, weight, and diagnosis. This information may allow monitor 14 to determine, for example, patient-specific threshold ranges in which the patient's physiological parameter measurements should fall and to enable or disable additional physiological parameter algorithms. Encoder 42 may, for instance, be a coded resistor which stores values corresponding to the type of sensor 12 or the type of each sensor in the sensor array, the wavelengths of light emitted by emitter 16 on each sensor of the sensor array, and/or the patient's characteristics. In another embodiment, encoder 42 may include a memory on which one or more of the following information may be stored for communication to monitor 14: the type of the sensor 12; the wavelengths of light emitted by emitter 16; the particular wavelength each sensor in the sensor array is monitoring; a signal threshold for each sensor in the sensor array; any other suitable information; or any combination thereof.

In an embodiment, signals from detector 18 and encoder 42 may be transmitted to monitor 14. In the embodiment shown, monitor 14 may include a general-purpose microprocessor 48 connected to an internal bus 50. Microprocessor 48 may be adapted to execute software, which may include an operating system and one or more applications, as part of performing the functions described herein. Also connected to bus 50 may be a read-only memory (ROM) 52, a random access memory (RAM) 54, user inputs 56, display 20, and speaker 22.

RAM 54 and ROM 52 are illustrated by way of example, and not limitation. Any suitable computer-readable media may be used in the system for data storage. Computer-readable media are capable of storing information that can be interpreted by microprocessor 48. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system.

In the embodiment shown, a time processing unit (TPU) 58 may provide timing control signals to a light drive circuitry 60, which may control when emitter 16 is illuminated and multiplexed timing for the RED LED 44 and the IR LED 46. TPU 58 may also control the gating-in of signals from detector 18 through an amplifier 62 and a switching circuit 64. These signals are sampled at the proper time, depending upon which light source is illuminated. The received signal from detector 18 may be passed through an amplifier 66, a low pass filter 68, and an analog-to-digital converter 70. The digital data may then be stored in a queued serial module (QSM) 72 (or buffer) for later downloading to RAM 54 as QSM 72 fills up. In one embodiment, there may be multiple separate parallel paths having amplifier 66, filter 68, and A/D converter 70 for multiple light wavelengths or spectra received.

In an embodiment, microprocessor 48 may determine the patient's physiological parameters, such as SpO₂ and pulse rate, using various algorithms and/or look-up tables based on the value of the received signals and/or data corresponding to the light received by detector 18. Signals corresponding to information about patient 40, and particularly about the intensity of light emanating from a patient's tissue over time, may be transmitted from encoder 42 to a decoder 74. These signals may include, for example, encoded information relating to patient characteristics. Decoder 74 may translate these signals to enable the microprocessor to determine the thresholds based on algorithms or look-up tables stored in ROM 52. User inputs 56 may be used to enter information about the patient, such as age, weight, height, diagnosis, medications, treatments, and so forth. In an embodiment, display 20 may exhibit a list of values which may generally apply to the patient, such as, for example, age ranges or medication families, which the user may select using user inputs 56.

The optical signal through the tissue can be degraded by noise, among other sources. One source of noise is ambient light that reaches the light detector. Another source of noise is electromagnetic coupling from other electronic instruments. Movement of the patient also introduces noise and affects the signal. For example, the contact between the detector and the skin, or the emitter and the skin, can be temporarily disrupted when movement causes either to move away from the skin. In addition, because blood is a fluid, it responds differently than the surrounding tissue to inertial effects, thus resulting in momentary changes in volume at the point to which the oximeter probe is attached.

Noise can degrade a pulse oximetry signal relied upon by a physician, without the physician's awareness. This is especially true if the monitoring of the patient is remote, the motion is too small to be observed, or the doctor is watching the instrument or other parts of the patient, and not the sensor site. Processing pulse oximetry (i.e., PPG) signals may involve operations that reduce the amount of noise present in the signals or otherwise identify noise components in order to prevent them from affecting measurements of physiological parameters derived from the PPG signals.

It will be understood that the present disclosure is applicable to any suitable signals and that PPG signals are used merely for illustrative purposes. Those skilled in the art will recognize that the present disclosure has wide applicability to other signals including, but not limited to other biosignals (e.g., electrocardiogram, electroencephalogram, electrogastrogram, electromyogram, heart rate signals, pathological sounds, ultrasound, or any other suitable biosignal), dynamic signals, non-destructive testing signals, condition monitoring signals, fluid signals, geophysical signals, astronomical signals, electrical signals, financial signals including financial indices, sound and speech signals, chemical signals, meteorological signals including climate signals, and/or any other suitable signal, and/or any combination thereof.

In one embodiment, a PPG signal may be transformed using a continuous wavelet transform. Information derived from the transform of the PPG signal (i.e., in wavelet space) may be used to provide measurements of one or more physiological parameters.

The continuous wavelet transform of a signal x(t) in accordance with the present disclosure may be defined as

$$T ( a , b ) = \frac{1}{\sqrt{a}} {\int }_{- ∞}^{+ ∞} x ( t ) {ψ}^{*} ( \frac{t - b}{a} ) d t \qquad ( 9 )$$

where ψ*(t) is the complex conjugate of the wavelet function ψ(t), a is the dilation parameter of the wavelet and b is the location parameter of the wavelet. The transform given by equation (9) may be used to construct a representation of a signal on a transform surface. The transform may be regarded as a time-scale representation. Wavelets are composed of a range of frequencies, one of which may be denoted as the characteristic frequency of the wavelet, where the characteristic frequency associated with the wavelet is inversely proportional to the scale a. One example of a characteristic frequency is the dominant frequency. Each scale of a particular wavelet may have a different characteristic frequency. The underlying mathematical detail required for the implementation within a time-scale can be found, for example, in Paul S. Addison, The Illustrated Wavelet Transform Handbook (Taylor & Francis Group 2002), which is hereby incorporated by reference herein in its entirety.

The continuous wavelet transform decomposes a signal using wavelets, which are generally highly localized in time. The continuous wavelet transform may provide a higher resolution relative to discrete transforms, thus providing the ability to garner more information from signals than typical frequency transforms such as Fourier transforms (or any other spectral techniques) or discrete wavelet transforms. Continuous wavelet transforms allow for the use of a range of wavelets with scales spanning the scales of interest of a signal such that small scale signal components correlate well with the smaller scale wavelets and thus manifest at high energies at smaller scales in the transform. Likewise, large scale signal components correlate well with the larger scale wavelets and thus manifest at high energies at larger scales in the transform. Thus, components at different scales may be separated and extracted in the wavelet transform domain. Moreover, the use of a continuous range of wavelets in scale and time position allows for a higher resolution transform than is possible relative to discrete techniques.

In addition, transforms and operations that convert a signal or any other type of data into a spectral (i.e., frequency) domain necessarily create a series of frequency transform values in a two-dimensional coordinate system where the two dimensions may be frequency and, for example, amplitude. For example any type of Fourier transform would generate such a two-dimensional spectrum. In contrast, wavelet transforms, such as continuous wavelet transforms, are required to be defined in a three-dimensional coordinate system and generate a surface with dimensions of time, scale and, for example, amplitude. Hence, operations performed in a spectral domain cannot he performed in the wavelet domain; instead the wavelet surface must be transformed into a spectrum (i.e., by performing an inverse wavelet transform to convert the wavelet surface into the time domain and then performing a spectral transform from the time domain). Conversely, operations performed in the wavelet domain cannot be performed in the spectral domain; instead a spectrum must first be transformed into a wavelet surface (i.e., by performing an inverse spectral transform to convert the spectral domain into the time domain and then performing a wavelet transform from the time domain). Nor does a cross-section of the three-dimensional wavelet surface along, for example, a particular point in time equate to a frequency spectrum upon which spectral-based techniques may be used. At least because wavelet space includes a time dimension, spectral techniques and wavelet techniques are not interchangeable. It will be understood that converting a system that relies on spectral domain processing to one that relies on wavelet space processing would require significant and fundamental modifications to the system in order to accommodate the wavelet space processing (e.g., to derive a representative energy value for a signal or part of a signal requires integrating twice, across time and scale, in the wavelet domain while, conversely, one integration across frequency is required to derive a representative energy value from a spectral domain). As a further example, to reconstruct a temporal signal requires integrating twice, across time and scale, in the wavelet domain while, conversely, one integration across frequency is required to derive a temporal signal from a spectral domain. It is well known in the art that, in addition to or as an alternative to amplitude, parameters such as energy density, modulus, phase, among others may all be generated using such transforms and that these parameters have distinctly different contexts and meanings when defined in a two-dimensional frequency coordinate system rather than a three-dimensional wavelet coordinate system. For example, the phase of a Fourier system is calculated with respect to a single origin for all frequencies while the phase for a wavelet system is unfolded into two dimensions with respect to a wavelet's location (often in time) and scale.

The energy density function of the wavelet transform, the scalogram, is defined as

S(a,b)=|T(a, b)|² (10)

where ‘∥’ is the modulus operator. The scalogram may be rescaled for useful purposes. One common rescaling is defined as

$${S}_{R} ( a , b ) = \frac{{\vert T ( a , b ) \vert}^{2}}{a} \qquad ( 11 )$$

and is useful for defining ridges in wavelet space when, for example, the Morlet wavelet is used. Ridges are defined as the locus of points of local maxima in the plane. Any reasonable definition of a ridge may be employed in the method. Also included as a definition of a ridge herein are paths displaced from the locus of the local maxima. A ridge associated with only the locus of points of local maxima in the plane are labeled a “maxima ridge”.

For implementations requiring fast numerical computation, the wavelet transform may be expressed as an approximation using Fourier transforms. Pursuant to the convolution theorem, because the wavelet transform is the cross-correlation of the signal with the wavelet function, the wavelet transform may be approximated in terms of an inverse FFT of the product of the Fourier transform of the signal and the Fourier transform of the wavelet for each required a scale and then multiplying the result by \sqrt{a}.

In the discussion of the technology which follows herein, the “scalogram” may be taken to include all suitable forms of rescaling including, but not limited to, the original unsealed wavelet representation, linear rescaling, any power of the modulus of the wavelet transform, or any other suitable rescaling. In addition, for purposes of clarity and conciseness, the term “scalogram” shall be taken to mean the wavelet transform, T(a,b) itself, or any part thereof. For example, the real part of the wavelet transform, the imaginary part of the wavelet transform, the phase of the wavelet transform, any other suitable part of the wavelet transform, or any combination thereof is intended to be conveyed by the term “scalogram”.

A scale, which may be interpreted as a representative temporal period, may be converted to a characteristic frequency of the wavelet function. The characteristic frequency associated with a wavelet of arbitrary a scale is given by

$$f = \frac{{f}_{c}}{a} \qquad ( 12 )$$

where f_{c}, the characteristic frequency of the mother wavelet (i.e., at a=1), becomes a scaling constant and f is the representative or characteristic frequency for the wavelet at arbitrary scale a.

Any suitable wavelet function may be used in connection with the present disclosure. One of the most commonly used complex wavelets, the Morlet wavelet, is defined as:

ψ(t)=π^{-1/4}(e^{i2πf}^{₀}^{t}−e^{-(2πf}^{₀}⁾^{^{>}}^{/2})e^{-t}^{²}^{/2} (13)

where f₀ is the central frequency of the mother wavelet. The second term in the parenthesis is known as the correction term, as it corrects for the non-zero mean of the complex sinusoid within the Gaussian window. In practice, it becomes negligible for values of f₀>>0 and can be ignored, in which case, the Morlet wavelet can be written in a simpler form as

$$ψ ( t ) = \frac{1}{{π}^{1 / 4}} {e}^{i 2 π {f}_{0} t} {e}^{- {t}^{2} / 2} \qquad ( 14 )$$

This wavelet is a complex wave within a scaled Gaussian envelope. While both definitions of the Morlet wavelet are included herein, the function of equation (14) is not strictly a wavelet as it has a non-zero mean (i.e., the zero frequency term of its corresponding energy spectrum is non-zero). However, it will be recognized by those skilled in the art that equation (14) may be used in practice with f₀>>0 with minimal error and is included (as well as other similar near wavelet functions) in the definition of a wavelet herein. A more detailed overview of the underlying wavelet theory, including the definition of a wavelet function, can be found in the general literature. Discussed herein is how wavelet transform features may be extracted from the wavelet decomposition of signals. For example, wavelet decomposition of PPG signals may be used to provide clinically useful information within a medical device.

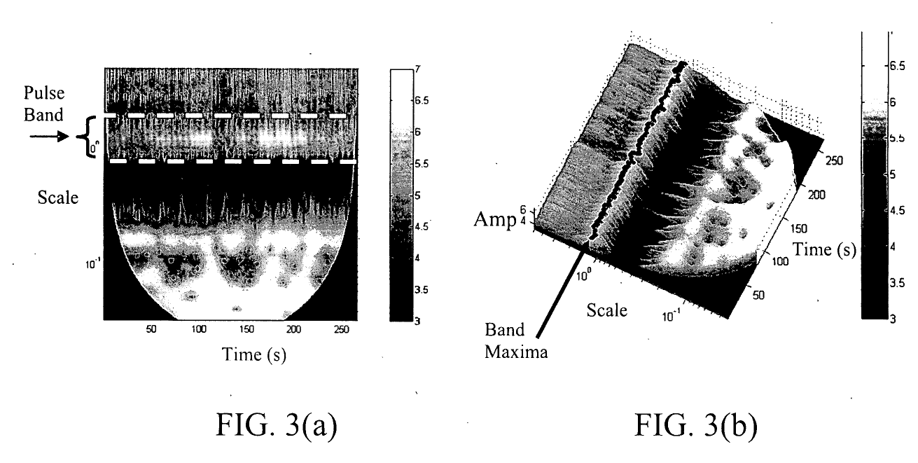

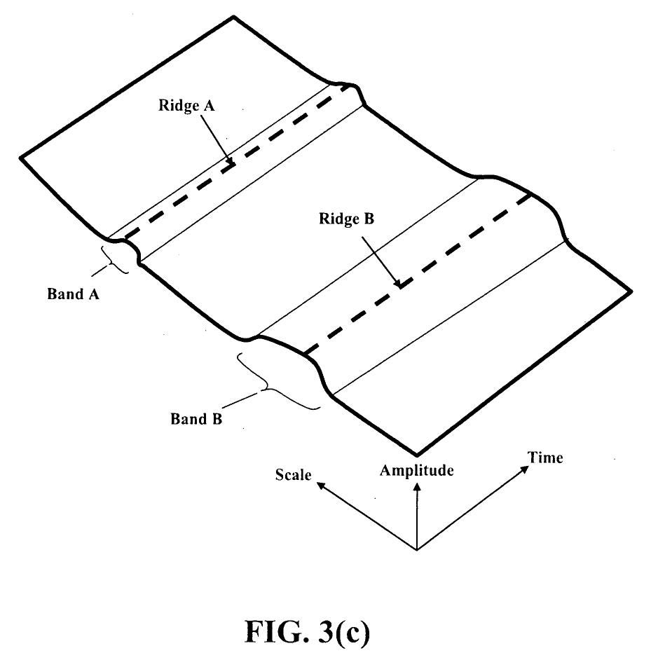

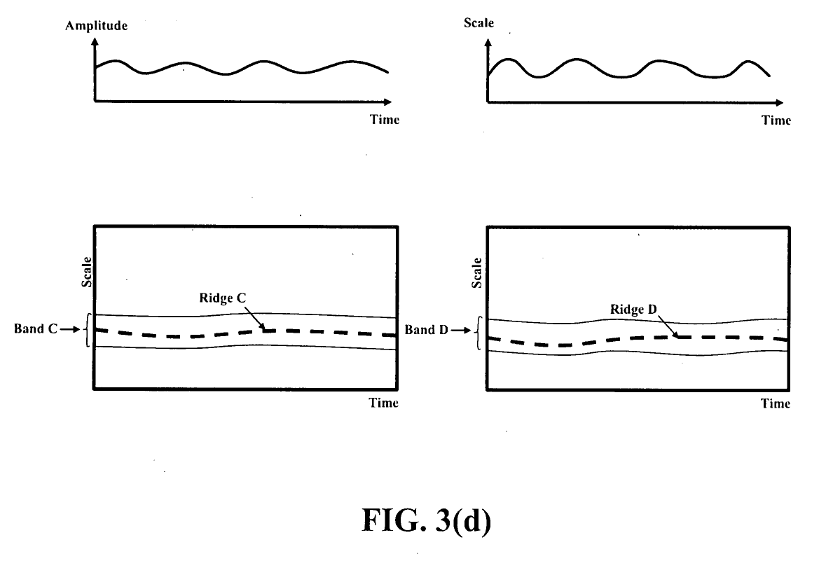

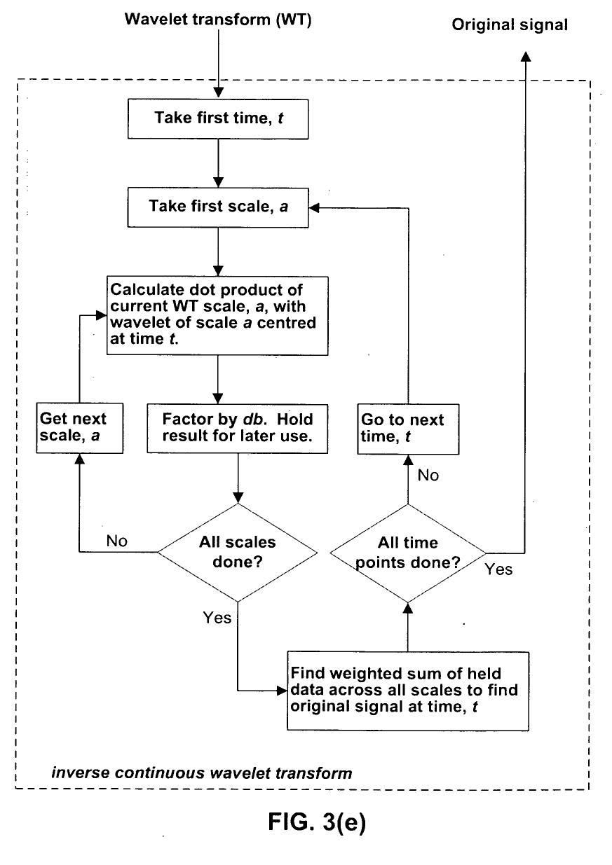

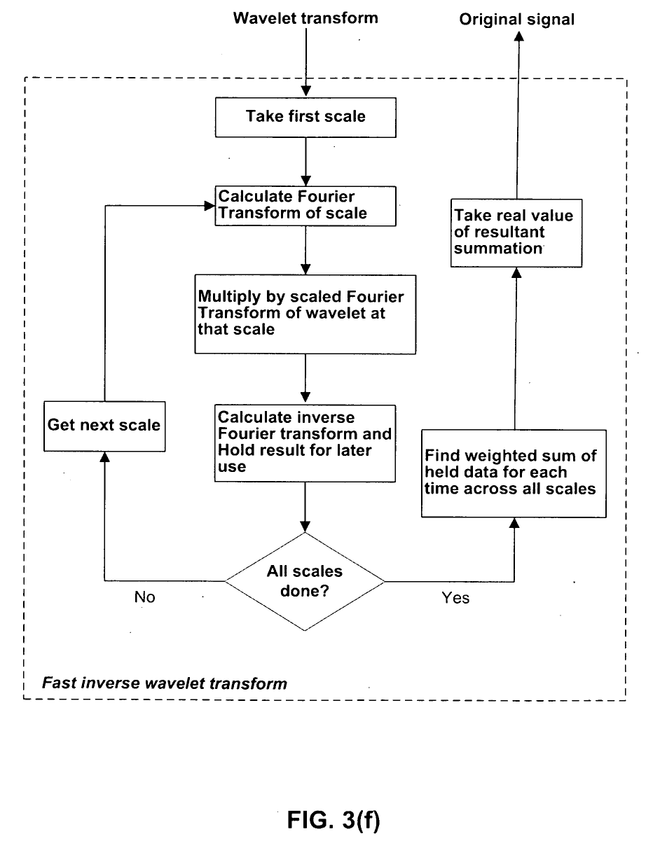

Pertinent repeating features in a signal give rise to a time-scale band in wavelet space or a rescaled wavelet space. For example, the pulse component of a PPG signal produces a dominant band in wavelet space at or around the pulse frequency. FIGS. 3(a) and (b) show two views of an illustrative scalogram derived from a PPG signal, according to an embodiment. The figures show an example of the band caused by the pulse component in such a signal. The pulse band is located between the dashed lines in the plot of FIG. 3(a). The band is formed from a series of dominant coalescing features across the scalogram. This can be clearly seen as a raised band across the transform surface in FIG. 3(b) located within the region of scales indicated by the arrow in the plot (corresponding to 60 beats per minute). The maxima of this band with respect to scale is the ridge. The locus of the ridge is shown as a black curve on top of the band in FIG. 3(b). By employing a suitable rescaling of the scalogram, such as that given in equation (11), the ridges found in wavelet space may be related to the instantaneous frequency of the signal. In this way, the pulse rate may be obtained from the PPG signal. Instead of rescaling the scalogram, a suitable predefined relationship between the scale obtained from the ridge on the wavelet surface and the actual pulse rate may also be used to determine the pulse rate.

By mapping the time-scale coordinates of the pulse ridge onto the wavelet phase information gained through the wavelet transform, individual pulses may be captured. In this way, both times between individual pulses and the timing of components within each pulse may be monitored and used to detect heart beat anomalies, measure arterial system compliance, or perform any other suitable calculations or diagnostics. Alternative definitions of a ridge may be employed. Alternative relationships between the ridge and the pulse frequency of occurrence may be employed.

As discussed above, pertinent repeating features in the signal give rise to a time-scale band in wavelet space or a rescaled wavelet space. For a periodic signal, this band remains at a constant scale in the time-scale plane. For many real signals, especially biological signals, the band may be non-stationary; varying in scale, amplitude, or both over time. FIG. 3(c) shows an illustrative schematic of a wavelet transform of a signal containing two pertinent components leading to two bands in the transform space, according to an embodiment. These bands are labeled band A and band B on the three-dimensional schematic of the wavelet surface. In this embodiment, the band ridge is defined as the locus of the peak values of these bands with respect to scale. For purposes of discussion, it may be assumed that band B contains the signal information of interest. This will be referred to as the “Primary band”. In addition, it may be assumed that the system from which the signal originates, and from which the transform is subsequently derived, exhibits some form of coupling between the signal components in band A and band B. When noise or other erroneous features are present in the signal with similar spectral characteristics of the features of band B then the information within band B can become ambiguous (i.e., obscured, fragmented or missing). In this case, the ridge of band A may be followed in wavelet space and extracted either as an amplitude signal or a scale signal which will be referred to as the “ridge amplitude perturbation” (RAP) signal and the “ridge scale perturbation” (RSP) signal, respectively. The RAP and RSP signals may be extracted by projecting the ridge onto the time-amplitude or time-scale planes, respectively. The top plots of FIG. 3(d) show a schematic of the RAP and RSP signals associated with ridge A in FIG. 3(c). Below these RAP and RSP signals are schematics of a further wavelet decomposition of these newly derived signals. This secondary wavelet decomposition allows for information in the region of band B in FIG. 3(c) to be made available as band C and band D. The ridges of bands C and D may serve as instantaneous time-scale characteristic measures of the signal components causing bands C and D. This technique, which will be referred to herein as secondary wavelet feature decoupling (SWFD), may allow information concerning the nature of the signal components associated with the underlying physical process causing the primary band B (FIG. 3(c)) to be extracted when band B itself is obscured in the presence of noise or other erroneous signal features.

In some instances, an inverse continuous wavelet transform may be desired, such as when modifications to a scalogram (or modifications to the coefficients of a transformed signal) have been made in order to, for example, remove artifacts. In one embodiment, there is an inverse continuous wavelet transform which allows the original signal to be recovered from its wavelet transform by integrating over all scales and locations, a and b:

$$x ( t ) = \frac{1}{{C}_{g}} {\int }_{- ∞}^{∞} {\int }_{0}^{∞} T ( a , b ) \frac{1}{\sqrt{a}} ψ ( \frac{t - b}{a} ) \frac{d a d b}{{a}^{2}} \qquad ( 15 )$$

which may also be written as:

$$x ( t ) = \frac{1}{{C}_{g}} {\int }_{- ∞}^{∞} {\int }_{0}^{∞} T ( a , b ) {ψ}_{a , b} ( t ) \frac{d a d b}{{a}^{2}} \qquad ( 16 )$$

where C_{g} is a scalar value known as the admissibility constant. It is wavelet type dependent and may be calculated from:

$${C}_{g} = {\int }_{0}^{∞} \frac{{\vert \hat{ψ} ( f ) \vert}^{2}}{f} d f \qquad ( 17 )$$

FIG. 3(e) is a flow chart of illustrative steps that may be taken to perform an inverse continuous wavelet transform in accordance with the above discussion. An approximation to the inverse transform may be made by considering equation (15) to be a series of convolutions across scales. It shall be understood that there is no complex conjugate here, unlike for the cross correlations of the forward transform. As well as integrating over all of a and b for each time t, this equation may also take advantage of the convolution theorem which allows the inverse wavelet transform to be executed using a series of multiplications. FIG. 3(f) is a flow chart of illustrative steps that may be taken to perform an approximation of an inverse continuous wavelet transform. It will be understood that any other suitable technique for performing an inverse continuous wavelet transform may be used in accordance with the present disclosure.

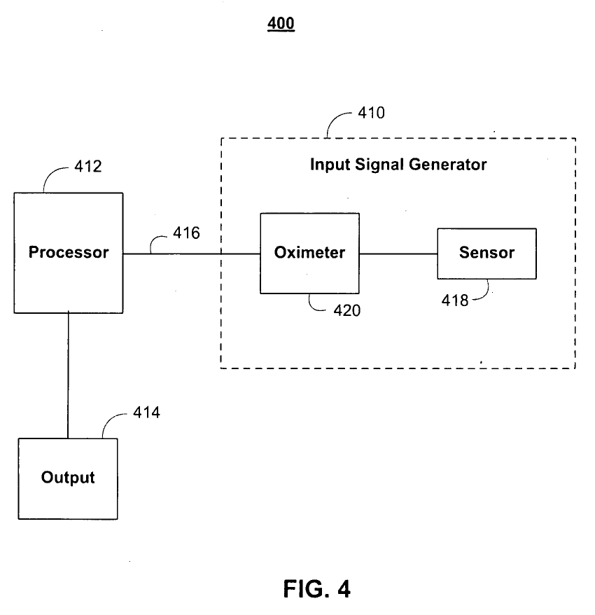

FIG. 4 is an illustrative continuous wavelet processing system in accordance with an embodiment. In this embodiment, input signal generator 410 generates an input signal 416. As illustrated, input signal generator 410 may include oximeter 420 coupled to sensor 418, which may provide as input signal 416, a PPG signal. It will be understood that input signal generator 410 may include any suitable signal source, signal generating data, signal generating equipment, or any combination thereof to produce signal 416. Signal 416 may be any suitable signal or signals, such as, for example, biosignals (e.g., electrocardiogram, electroencephalogram, electrogastrogram, electromyogram, heart rate signals, pathological sounds, ultrasound, or any other suitable biosignal), dynamic signals, non-destructive testing signals, condition monitoring signals, fluid signals, geophysical signals, astronomical signals, electrical signals, financial signals including financial indices, sound and speech signals, chemical signals, meteorological signals including climate signals, and/or any other suitable signal, and/or any combination thereof.

In this embodiment, signal 416 may be coupled to processor 412. Processor 412 may be any suitable software, firmware, and/or hardware, and/or combinations thereof for processing signal 416. For example, processor 412 may include one or more hardware processors (e.g., integrated circuits), one or more software modules, computer-readable media such as memory, firmware, or any combination thereof. Processor 412 may, for example, be a computer or may be one or more chips (i.e., integrated circuits). Processor 412 may perform the calculations associated with the continuous wavelet transforms of the present disclosure as well as the calculations associated with any suitable interrogations of the transforms. Processor 412 may perform any suitable signal processing of signal 416 to filter signal 416, such as any suitable band-pass filtering, adaptive filtering, closed-loop filtering, and/or any other suitable filtering, and/or any combination thereof.

Processor 412 may be coupled to one or more memory devices (not shown) or incorporate one or more memory devices such as any suitable volatile memory device (e.g., RAM, registers, etc.), non-volatile memory device (e.g., ROM, EPROM, magnetic storage device, optical storage device, flash memory, etc.), or both. The memory may be used by processor 412 to, for example, store data corresponding to a continuous wavelet transform of input signal 416, such as data representing a scalogram. In one embodiment, data representing a scalogram may be stored in RAM or memory internal to processor 412 as any suitable three-dimensional data structure such as a three-dimensional array that represents the scalogram as energy levels in a time-scale plane. Any other suitable data structure may be used to store data representing a scalogram.

Processor 412 may be coupled to output 414. Output 414 may be any suitable output device such as, for example, one or more medical devices (e.g., a medical monitor that displays various physiological parameters, a medical alarm, or any other suitable medical device that either displays physiological parameters or uses the output of processor 412 as an input), one or more display devices (e.g., monitor, PDA, mobile phone, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

It will be understood that system 400 may be incorporated into system 10 (FIGS. 1 and 2) in which, for example, input signal generator 410 may be implemented as parts of sensor 12 and monitor 14 and processor 412 may be implemented as part of monitor 14.

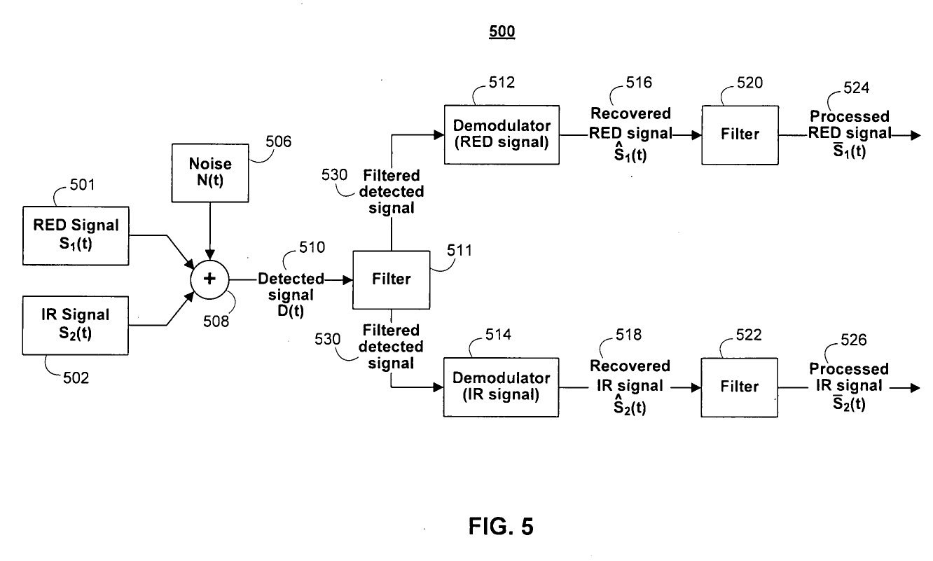

FIG. 5 shows an illustrative module 500 for reducing noise and cross-talk in one or more detected PPG signals. Cross-talk may refer to a phenomenon in a measurement system in which two signals cause mutual interference to each other and thus may degrade the quality of the overall measured signal. Module 500 may be incorporated as a component of system 10 (FIGS. 1 and 2), system 400, or any other suitable system. In an embodiment, module 500 may process signals detected by a detector such as detector 18 (FIG. 1) to remove generally undesirable artifacts such as cross-talk and additive noise. Such pre-processing may be performed in, for example, a monitor such as monitor 14 (FIG. 1) using a processor such as processor 412 (FIG. 4) or microprocessor 48 (FIG. 2). After processing by module 500, processed versions of the detected signals may be used to determine one or more physiological parameters of patient 40 (FIG. 2). For example, one or more processed versions of the detected signals may be analyzed using wavelet transform techniques and/or one or more scalograms to determine, for example, pulse rate, respiration rate, and/or blood oxygen saturation of patient 40 (FIG. 2).

Referring back to FIGS. 1 and 2, signals emitted by emitter 18 (FIG. 1) may include radiation emitted by RED LED 44 (FIG. 2) and/or radiation emitted by IR LED 46 (FIG. 2). In an embodiment, detected signal 510 (e.g., the signal detected by detector 18 (FIG. 1)) may include detected RED signal 501, detected IR signal 502, and noise signal 506. In an embodiment, detected signal 510 may be represented mathematically as the addition 508 of these individual signals, as described by the following relationship

D(t)=S₁(t)+S₂(t)+N(t), (18)

where D(t) refers to detected signal 510, S₁(t) refers to detected RED signal 501, S₂(t) refers to the detected IR signal 502, and N(t) refers to (generally undesirable) system noise 506. Each of the signals in equation (18) includes the index t to emphasize that each signal may generally be a function of time

In an embodiment, detected RED signal 501 may correspond to the detected physiological response of patient 40 (FIG. 2) to a pulsed RED signal emitted by emitter 16 (FIG. 1). For example, the emitted signal may be a periodic pulse train having a duty cycle of a certain value. The duty cycle of detected RED signal 501 may be 10-percent, 20-percent, 25-percent, or any other suitable percentage, where duty-cycle refers to the percentage of time that RED light is emitted by RED LED 44 (FIG. 2). In an embodiment, detected IR signal 502 may be the detected physiological response of patient 40 (FIG. 2) to a pulsed IR signal emitted by emitter 16 (FIG. 1). For example, the emitted signal may be a periodic pulse train having a duty cycle of a certain value. The duty cycle of detected IR signal 502 may be 10-percent, 20-percent, 25-percent, or any other suitable percentage, where the duty-cycle refers to the percentage of time that IR light is emitted by IR LED 46 (FIG. 2). In an embodiment, both detected RED signal 501 and detected IR signal 502 may correspond to the physiological response of patient 40 (FIG. 2) to two pulsed signals, each of a certain duty cycle. In an embodiment, detected RED signal 501 and detected IR signal 502 may be characterized by possibly noisy pulses that do not overlap in time. Alternatively, detected RED signal 501 and detected IR signal 502 may be characterized by possibly noisy pulses that overlap partially or fully in time. Noise 506 may correspond to the effects of the undesirable noise detected by detector 18 (FIG. 1). Noise 506 may include the effects of thermal noise, shot noise, flicker noise, burst noise, and electrical noise caused by light pollution, or any other suitable type of noise, either individually or in any suitable combination.

In an embodiment, detected signal 510 may be filtered using filter 511. Filter 511 may be implemented in monitor 14 (FIG. 1), for example, using processor 412 (FIG. 4) or microprocessor 48 (FIG. 2). Filter 511 may correspond to a band-pass filter, a low-pass filter, or any other suitable filter. Filter 511 may be an analog filter, or any other suitable type of filter. In an embodiment, filter 511 may be designed to eliminate or substantially reduce noise 506 present in detected signal 510. For example, filter 511 may be a low-pass or band-pass filter that admits only frequencies corresponding to detected RED signal 501 and detected IR signal 502. Such a low-pass or band-pass filter may substantially reduce the effect of noise 506 in detected signal 510 by filtering out-of-band noise components. In an embodiment, module 500 may include an analog-to-digital converter (not shown) and/or a sampling and decimation filter (not shown). These components may be included as part of filter 511 or may be used prior to or after filter 511 as part of module 500. Filter 511 may process detected signal 510 to produce filtered detected signal 530.

In an embodiment, filtered detected signal 530 may be processed separately by demodulator 512 and demodulator 514. Demodulator 512 may process filtered detected signal 530 to recover a component of detected signal 510 corresponding to detected RED signal 501, and demodulator 514 may process filtered detected signal 530 to recover the component of detected signal 510 corresponding to detected IR signal 502.

In an embodiment, demodulator 512 may apply a filter to filtered detected signal 530 to recover the component of filtered detected signal 530 corresponding to detected RED signal 501. Alternatively, demodulator 512 may multiply filtered detected signal 530 by a suitable window function to extract and hence recover the component of filtered detected signal 530 corresponding to detected RED signal 501. In an embodiment, recovered RED signal 516 may be represented mathematically by the following equation

Ŝ₁(t)=S₁(t)+β₁(t)S₂(t), (19)

where Ŝ₁(t) refers to recovered RED signal 516, β₁(t) represents cross-talk in recovered RED signal 516, and where S₁(t) and S₂(t) were described previously in equation (18). Cross-talk β₁(t) in equation (19) corresponds to the presence of detected IR signal 502 in recovered RED signal 516. Cross-talk may be introduced by many sources, either individually or in combination. For example, in module 500, filter 511 or demodulator 512 may cause cross-talk. In system 10 (FIGS. 1 and 2), electrical malfunction in sensor 12 (FIG. 1), detector 18 (FIG. 1), emitter 16 (FIG. 1), or a cable such as cable 24 (FIG. 1) may cause cross-talk. The cross-talk represented in equation (19) may include components from each of these sources as well as other electrical and mechanical sources not pictured in FIGS. 1-5. In an embodiment, filter 511 contains an analog band-pass filter and the analog band-pass filter introduces cross-talk to filtered detected signal 530.

In an embodiment, demodulator 514 may apply a filter to filtered detected signal 530 to recover the component of filtered detected signal 530 corresponding to detected IR signal 502. Alternatively, demodulator 514 may multiply filtered detected signal 530 by a suitable window function to extract, and hence recover, the component of filtered detected signal 530 corresponding to detected IR signal 502. In an embodiment, recovered IR signal 518 may be represented mathematically by the following equation

Ŝ₂(t)=S₂(t)+β₂(t)S₁(t), (20)

where Ŝ₂(t) refers recovered IR signal 518, β₂(t) represents cross-talk in recovered IR signal 518, and where S₁(t) and S₂(t) were described previously in equation (18). Cross-talk β₂(t) in equation (20) corresponds to the presence of detected RED signal 501 in recovered IR signal 518. Cross-talk may be caused by many sources, either individually or in combination. For example, in module 500, filter 511 or demodulator 514 may cause cross-talk. In system 10 (FIGS. 1 and 2), electrical malfunction in sensor 12 (FIG. 1), detector 18 (FIG. 1), emitter 16 (FIG. 1), or a cable such as cable 24 (FIG. 1) may cause cross-talk. The cross-talk represented in equation (20) may include components from each of these sources as well as other electrical and mechanical sources not pictured in FIGS. 1-5. In an embodiment, filter 511 contains an analog band-pass filter and the analog band-pass filter introduces cross-talk to filtered detected signal 530.

In an embodiment, recovered RED signal 516 may be processed by filter 520 to obtain processed RED signal 524. Filter 520 may be designed to partially or fully remove cross-talk present in recovered RED signal 516, e.g., as described in equation (19). For example, filter 520 may be a low-pass filter digital filter of order two, three, four, or any other suitable order, and the coefficients of filter 520 may be selected to partially or fully remove cross-talk from recovered RED signal 516. Filter 520 may incorporate any suitable filter design technique to remove cross-talk. For example, filter 520 may use well-known filter design techniques from signal processing such as order-matching and transfer-function analysis techniques. In an embodiment, filter 520 may contain additional low-pass or band-pass filters. In an embodiment, processed RED signal 524 may be represented mathematically by the following equation

S̅₁(t)=S₁(t)+ β̅₁(t)S₂(t), (21)

where S̅₁(t) refers to processed RED signal 524, β̅₁(t) represents residual cross-talk in processed RED signal 524, and where S₁(t) and S₂(t) were described previously in equation (18). In an embodiment, filter 520 may completely remove cross-talk from processed RED signal 524. In such an embodiment, β̅₁(t)=0 at all times t.

In an embodiment, recovered IR signal 518 may be processed by filter 522 to obtain processed IR signal 526. Filter 522 may be designed to partially or fully remove cross-talk present in recovered IR signal 518, e.g., as described in equation (20). For example, filter 522 may be a low-pass filter digital filter of order two, three, four, or any other suitable order, and the coefficients of filter 522 may be selected to partially or fully remove cross-talk from recovered RED signal 518. Filter 522 may incorporate any suitable filter design technique to remove cross-talk. For example, filter 522 may use well-known filter design techniques from signal processing such as order-matching and transfer-function analysis techniques. In an embodiment, filter 522 may contain additional low-pass or band-pass filters. In an embodiment, processed IR signal 526 may be represented mathematically by the following equation

S̅₂(t)=S₂(t)+ β̅₂(t)S₁(t), (22)

where S̅₂(t) refers to processed IR signal 526, β₂(t) represents residual cross-talk in processed IR signal 526, and where S₁(t) and S₂(t) were described previously in equation (18). In an embodiment, filter 522 may completely remove cross-talk from processed IR signal 526. In such an embodiment, β̅₂(t)=0 at all times t.

In an embodiment, processed RED signal 524 and processed IR signal 526 may be used individually or in combination to determine or estimate physiological characteristics of patient 40 (FIG. 2). In an embodiment, cross-talk is partially or fully removed from processed RED signal 524 and processed IR signal 526, and this may result in more reliable determination or estimation of physiological characteristics than would be possible in a counterpart system that did not remove cross-talk from the signal detected by, for example, detector 18 (FIG. 1). Processed RED signal 524 and processed IR signal 526 may be analyzed using wavelet transform techniques and/or one or more scalograms. Such an analysis may be carried out by a system such as system 400 (FIG. 4) or system 10 (FIGS. 1 and 2) using continuous wavelet transform (CWT) techniques, as described in relation to FIGS. 1-4.

In an embodiment, systems 400 (FIG. 4) and 10 (FIGS. 1 and 2) may use CWT techniques for calculating pulse rate of patient 40 (FIG. 2). For example, the pulse component of a PPG signal may produce a dominant band in a scalogram. The pulse rate may be calculated, for example, using a CWT technique by generating a scalogram from one or more PPG signals, following or identifying the ridge of the pulse band, identifying a scale corresponding to the ridge, and selecting the pulse rate to be the characteristic frequency of the identified scale. In an embodiment, the one or more PPG signals may include processed RED signal 524 and processed IR signal 526, and these signals may be acquired using a module such as module 500. A scalogram generated from such signals may be of higher quality (for example, as measured by the signal-to-noise level of the scalogram) than would be possible if module 500 was not used to remove noise and cross-talk. Thus, module 500 may provide a more accurate determination of pulse rate of patient 40. For example, identifying the ridge of pulse band and identifying a scale corresponding to the ridge may be done more accurately when module 500 is used than when it is not.

In an embodiment, systems 400 (FIG. 4) and 10 (FIGS. 1 and 2) may use CWT techniques for calculating respiration rate of patient 40 (FIG. 2). The respiration component of a PPG signal may produce a band in a scalogram similar to the pulse band. Therefore, the respiration rate may be calculated, for example, by generating a scalogram from one or more PPG signals, following or identifying the ridge of the respiration band (e.g., located at scales lower than the scales where the typically more dominant pulse band occurs), identifying a scale corresponding to the ridge, and selecting the respiration rate to be the characteristic frequency of the identified scale. In an embodiment, the one or more PPG signals may include processed RED signal 524 and processed IR signal 526, and these signals may be acquired using a module such as module 500. A scalogram generated from such signals may be of higher quality (for example, as measured by the signal-to-noise level of the scalogram) than would be possible if module 500 was not used to remove noise and cross-talk. Thus, module 500 may provide a more accurate determination of respiration rate of patient 40. For example, identifying the ridge of the respiration band and identifying a scale corresponding to the ridge may be done more accurately when module 500 is used than when it is not. The respiration component of the PPG signal may also cause modulations of the pulse band. Thus, the respiration rate may also be calculated by performing a secondary wavelet decomposition of modulations (erg, of RAP and RSP signals) of the pulse band. These and other CWT techniques for calculating respiration rate are described in detail in Addison et al. U.S. Pat. No. 7,035,679, Addison et al. U.S. Patent Publication No. 2006/0258921, and U.S. patent application Ser. No. ______, filed Oct. 3, 2008, entitled “Systems And Methods For Ridge Selection In Scalograms Of Signals,” (Att. Docket No.: H-RM-01197-1 (COV-2-01)), each of which is hereby incorporated by reference herein in its entirety.

In an embodiment, systems 400 (FIG. 4) and 10 (FIGS. 1 and 2) may use CWT techniques for calculating oxygen saturation of patient 40 (FIG. 2). Oxygen saturation may be determined, for example, by computing the ratio of points on two scalograms (e.g., at the location of the pulse band) and using, for example, a lookup table or an equation to obtain oxygen saturation. Another continuous wavelet transform-based technique for calculating blood oxygen saturation involves generating a Lissajous figure in which transformed processed RED signal 524 and processed IR signal 526 may be plotted with respect to one another. Processed RED signal 524 and processed IR signal 526 may be acquired using a module such as module 500. A scalogram generated from such signals may be of higher quality (for example, as measured by the signal-to-noise level of the scalogram) than would be possible if module 500 was not used to remove noise and cross-talk. Thus, module 500 may provide a more accurate determination of blood oxygen saturation of patient 40. For example, computing the ratio of points on two scalograms may be done more accurately when module 500 is used than when it is not. These CWT techniques and other CWT techniques for determining oxygen saturation are described in detail in Addison et al. U.S. Patent App. Pub. No. 2006/0258921. Another exemplary CWT technique that may be used in accordance with this disclosure is described in U.S. patent application Ser. No. ______, filed Oct. 3, 2008, entitled “METHODS AND SYSTEMS FOR FILTERING A SIGNAL ACCORDING TO A SIGNAL MODEL AND CONTINUOUS WAVELET TRANSFORM TECHNIQUES,” (Att. Docket No. H-RM-01256-1 (COV-20-01)), which is hereby incorporated by reference herein in its entirety. This CWT technique includes generating a plurality of possible values in accordance with a signal model and determining which of the values has a highest energy level (e.g., to minimize correlation), and other techniques.

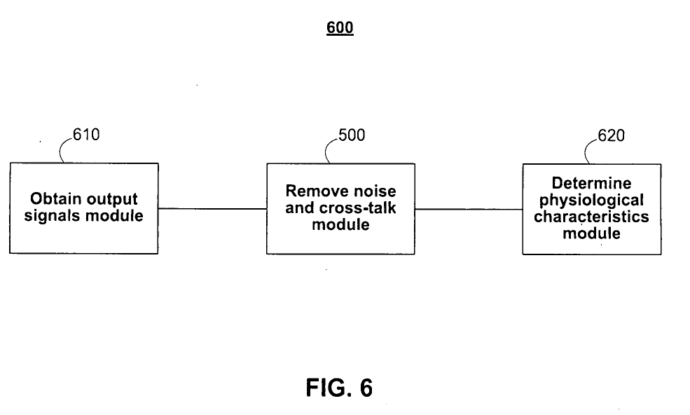

FIG. 6 shows an illustrative system for obtaining two or more output signals, removing noise and cross talk from the two or more output signals to produce processed output signals, and determining physiological characteristics based on processed output signals. System 600 may include modules 610, 500, and 620. In an embodiment, module 610 may be implemented by sensor 20 (FIG. 1). Module 610 may obtain two or more output signals which may correspond to two or more PPG signals. In an embodiment, two or more wavelengths of radiation are emitted by an emitter such as emitter 16 (FIG. 1). After passing through patient's 40 (FIG. 2) tissue, the two or more wavelengths of radiation are detected by detector 18 (FIG. 1). Detector 18 (FIG. 1) may convert the detected radiation into two or more respective electrical output signals. In an embodiment, the two or more output signals may include detected RED signal 501 (FIG. 5) and/or a detected IR signal 502 (FIG. 5).

Referring back to FIG. 6, the two or more output signals may be processed by a module such as module 500 (FIG. 5 and FIG. 6) to partially or fully remove noise and cross-talk from the two or more output signals. Module 500 may be implemented as described in relation to FIG. 5 In an embodiment, module 500 may produce two or more processed output signals based on the two or more output signals. For example, module 500 may filter and demodulate detected signal 510 (FIG. 5) to generate the two or more processed output signals. In an embodiment, the two or more processed output signals may include processed RED signal 524 (FIG. 5) and/or a processed IR signal 526 (FIG. 5).

Referring back to FIG. 6, after being processed by module 500, the two or more processed output signals may be sent to module 620. Module 620 may be implemented using system 400 (FIG. 4) or system 10 (FIGS. 1 and 2) and the techniques described in FIG. 3. In an embodiment, module 620 may compute one or more continuous wavelet transforms associated with the two or more processed output signals, generate one or more scalograms corresponding to the one or more wavelet transforms, and analyze the one or more scalograms to determine physiological characteristics of patient 40 (FIG. 2). In an embodiment, the two or more processed output signals may include processed RED signal 524 (FIG. 5) and processed IR signal 526 (FIG. 5). In an embodiment, module 620 may more accurately determine physiological characteristics of patient 40 (FIG. 2) than would be possible in a counterpart system that did not include module 500 to partially or fully remove noise and cross-talk in the two or more output signals.

The foregoing is merely illustrative of the principles of this disclosure and various modifications can be made by those skilled in the art without departing from the scope and spirit of the disclosure. The following claims may also describe various aspects of this disclosure.

What is claimed is:

1. A method of determining one or more physiological characteristics in a measurement system comprising:
detecting at least two wavelengths of radiation at a detector, the detector outputting at least two respective output signals;
processing the at least two output signals to reduce cross-talk to produce at least two processed signals;
transforming at least one of the at least two processed signals at least in part using a continuous wavelet transform;
generating a scalogram based at least in part on the wavelet transform; and
analyzing one or more characteristics of the scalogram to determine one or more physiological characteristics.

2. The method of claim 1, wherein processing the at least two output signals comprises:
summing the at least two output signals and a noise signal to obtain a detected signal;
filtering the detected signal to obtain a filtered detected signal;
demodulating the filtered detected signal to obtain a first recovered signal;
demodulating the filtered detected signal to obtain a second recovered signal;
filtering the first recovered signal to obtain a first processed signal; and
filtering the second recovered signal to obtain a second processed signal.

3. The method of claim 1, wherein the at least two output signals comprise a infrared signal and a red signal.

4. The method of claim 1, wherein the one or more physiological characteristics includes physiological characteristics selected from the group consisting of: a pulse rate; a respiration rate; and a blood oxygen saturation.

5. The method of claim 2, wherein filtering the first recovered signal comprises applying a low-pass filter to the first recovered signal and filtering the second recovered signal comprises applying a low-pass filter to the second recovered signal.

6. The method of claim 2, wherein filtering the detected signal comprises applying an analog band-pass filter to the detected signal.

7. The method of claim 2, wherein filtering the detected signal reduces noise present in the detected signal.

8. The method of claim 2, wherein filtering the detected signal generates cross-talk in the filtered detected signal.

9. The method of claim 5, wherein the low-pass filter applied to the first recovered signal includes filter coefficients selected at least in part to reduce cross-talk in the first recovered signal and wherein the low-pass filter applied to the second recovered signal includes filter coefficients selected at least in part to reduce cross-talk in the second recovered signal.

10. A system for determining one or more physiological characteristics in a measurement system comprising:
a sensor capable of:
detecting at least two wavelengths of radiation at a detector, and
outputting at least two respective output signals; and
a processor capable of:
processing the at least two output signals to reduce cross-talk to produce at least two processed signals,
transforming at least one of the at least two processed signals at least in part using a continuous wavelet transform,
generating a scalogram based at least in part on the wavelet transform, and
analyzing one or more characteristics of the scalogram to determine one or more physiological characteristics.

11. The system of claim 10, wherein processing the at least two output signals comprises:
summing the at least two output signals and a noise signal to obtain a detected signal;
filtering the detected signal to obtain a filtered detected signal;
demodulating the filtered detected signal to obtain a first recovered signal;
demodulating the filtered detected signal to obtain a second recovered signal;
filtering the first recovered signal to obtain a first processed signal; and
filtering the second recovered signal to obtain a second processed signal.

12. The system of claim 11, wherein the at least two output signals comprise a infrared signal and a red signal.

13. The system of claim 12, wherein the one or more physiological characteristics includes physiological characteristics selected from the group consisting of: a pulse rate; a respiration rate; and a blood oxygen saturation.

14. The system of claim 12, wherein filtering the first recovered signal comprises applying a low-pass filter to the first recovered signal and filtering the second recovered signal comprises applying a low-pass filter to the second recovered signal.

15. The system of claim 12, wherein filtering the detected signal comprises applying an analog band-pass filter to the detected signal.

16. The system of claim 12, wherein filtering the detected signal reduces noise present in the detected signal.

17. The system of claim 12, wherein filtering the detected signal generates cross-talk in the filtered detected signal.

18. The system of claim 15, wherein the low-pass filter applied to the first recovered signal includes filter coefficients selected at least in part to reduce cross-talk in the first recovered signal and wherein the low-pass filter applied to the second recovered signal includes filter coefficients selected at least in part to reduce cross-talk in the second recovered signal.

19. A computer-readable medium for use in determining one or more physiological characteristics in a measurement system, the computer-readable medium having computer program instructions recorded thereon for:
detecting at least two wavelengths of radiation at a detector;
the detector outputting at least two respective output signals;
processing the at least two output signals to reduce cross-talk to produce at least two processed signals;
transforming at least one of the at least two processed signals at least in part using a continuous wavelet transform;
generating a scalogram based at least in part on the wavelet transform; and
analyzing one or more characteristics of the scalogram to determine one or more physiological characteristics.

20. The computer-readable medium of claim 19, having further computer program instructions for:
summing the at least two output signals and a noise signal to obtain a detected signal;
filtering the detected signal to obtain a filtered detected signal;
demodulating the filtered detected signal to obtain a first recovered signal;
demodulating the filtered detected signal to obtain a second recovered signal;
filtering the first recovered signal to obtain a first processed signal; and
filtering the second recovered signal to obtain a second processed signal.
